(19) United States
(12) Patent Application Publication
(10) Pub. No.: US 2024/0315294 A1
(43) Pub. Date: Sep. 26, 2024

(54) METHODS AND PROCESSES FOR CULTURING CELLS

(71) Applicant: CELL AG TECH INC, York (CA)

(72) Inventors: Valentin FULGA, Toronto (CA); Simon BUBNIC, Scarborough (CA); Elizabeth JOHNSTON, Guelph (CA); Haidun LIU, Brampton (CA); Majid EBRAHIMI, Toronto (CA); Joshua POLLACK, Etobicoke (CA); Peter CURRIE, Melbourne (AU)

(21) Appl. No.: 18/259,987

(22) PCT Filed: Dec. 22, 2021
(86) PCT No.: PCT/CA2021/051879

§ 371 Date: Jun. 29, 2023

Related U.S. Application Data
(60) Provisional application No. 63/132,084, filed on Dec. 30, 2020.

(51) Int. Cl. A23L 17/00; C12N 5/077

(52) U.S. Cl. CPC A23L 17/00; C12N 5/0658; C12N 2501/105; C12N 2501/115; C12N 2509/00; C12N 2533/54

(57) ABSTRACT

Cultivated seafood muscle cells are produced through a process that first isolates culture initiating cells from tissue using mechanical and enzymatic means and then expanding their number over time in culture. Methods for enhancing yield of seafood muscle cells are used for manufacturing a seafood product.

### CROSS-REFERENCE TO RELATED APPLICATIONS

The application claims the benefit of U.S. provisional application No. 63/132,084, filed on Dec. 30, 2020, the entire content of which is incorporated herein by reference.

### FIELD

The present disclosure generally relates to cultured cells, foodstuffs and meats, including seafood.

### BACKGROUND

Cellular agriculture/aquaculture is an emerging industry aimed at addressing the difficulties that modern food production has started to encounter and will become more acute as the global population grows which is expected to reach approximately 10 billion in 2050. This industry attempts to produce animal protein in a more sustainable way by reducing the environmental impact of modern agriculture and improving animal welfare.

Instead of growing the entire animal for food, cellular agriculture/aquaculture uses stem cells (primarily satellite cells or myoblasts) grown and expanded in culture as the raw material to produce meat for consumption. While scientifically this is a viable idea, there are a number of technological and financial challenges. Scale-up, cost of goods, energy consumption, building a 3D construct similar in structure and texture to natural meat, and maintaining flavour are some of the main challenges that a successful cell-based food product or cultured meat must overcome.

In particular, culturing seafood meat remains a major challenge.

### SUMMARY

In one aspect, a method is provided for enhancing the yield of progenitor muscle cells cultured from a sample cell culture, the sample cell culture comprising a plurality of cell types in a culture medium, the method comprising: (a) plating the sample cell culture on a first surface; (b) culturing the plated cell culture for sufficient time to allow the progenitor muscle cells to attach onto the first surface; (c) collecting at least a portion of the culture medium, comprising unattached progenitor muscle cells; and (d) replating the collected culture medium from (c) onto a further surface to allow the unattached progenitor muscle cells to attach onto the further surface.

In another aspect, a method of producing a food product is provided, the method comprising: a) extracting cells from a tissue sample obtained from an organism, or from a body part of the organism, post-mortem; and b) isolating and culturing progenitor muscle cells from the tissue sample into a food product.

In another aspect, a method is provided for isolating progenitor muscle cells from a tissue sample comprising separating the progenitor muscle cells from at least one other cell or cellular debris using at least one density gradient.

In yet another aspect, a food product is provided made by the method described herein, the food product comprising: a) first fish muscle cells; and b) seafood cells. In yet another aspect, a food product is provided made by the method described herein, the food product comprising: a) cultured fish muscle cells, either alone or in combination with b) other cultured cells, c) animal derived ingredients and/or d) non-animal derived ingredients.

Many further features and combinations thereof concerning embodiments described herein will appear to those skilled in the art following a reading of the instant disclosure.

### DESCRIPTION OF THE FIGURES

Having thus generally described the nature of the invention, reference will now be made to the accompanying drawings, showing by way of illustration, a preferred embodiment thereof, and in which:

FIG. 1 shows a graph of the yield of cultured cells derived from tilapia stored for 1 to 17 days post-mortem. The data are culture yields normalized to the amount of tissue processed arising from single experiments at each time point.

FIG. 2 shows bar graphs of the yield of cultured cells. A) shows yields of cultured cells following treatment of tissue with collagenase and TrypLE. Cells were collected after the TrypLE step only (first bar from left, medium blue, n=3), or after both collagenase and TrypLE incubations (second bar from left grey, n=2), or after subsequent density gradient centrifugation with Ficoll-Paque alone (third bar from left light blue, n=6), or after sequential Ficoll-Paque and Percoll gradients (fourth bar from left, dark blue, n=4). B) shows data from an experiment directly comparing cell yield when cultures were initiated using cells after enzyme incubation versus those initiated with cells after Ficoll-Paque density gradient separation.

FIG. 3 shows images from day two of muscle cell cultures. The image on the left shows day two of muscle cell cultures initiated directed after enzyme treatment. The image on the right shows day two of muscle cell cultures initiated after Ficoll-Paque density gradient separation.

FIG. 4 shows images of original and serial reseeded cultures taken on the same day. The panel on the left shows the original cultures taken on its day 6. The panel in the middle shows the first reseeded cultures taken on its day 5. The panel on the right shows the second reseeded cultures taken on its day 4. Adherent muscle cells are visible in all three cultures.

FIG. 5 shows an image of day 1 after initial seeding of culture. Settling cells are visible as primarily round in shape.

FIG. 6 shows an image of the distinctive morphology of myoblasts (spindle-shaped) and fibroblasts (irregular shape) within the first few days of culture. Example myoblasts and fibroblasts are indicated with black arrows. The image was taken from a primary culture from day 4.

FIG. 7 shows an image of actively growing culture exhibiting approximately—70-80% confluence. The image was obtained from a primary culture from day 9.

FIG. 8 shows an image of the expression of Pax3 in nuclei of primary culture (T21). The white arrows indicate the regions of the image demonstrating Pax3 expression. All nuclei are stained with DAPI (imaged in blue), while Pax3 staining is visualized by red fluorescence resulting in reddish to purple nuclei.

FIG. 9 shows an image of the expression of desmin (imaged in red) in first passage cell culture (T23). Nuclei are stained with DAPI (imaged in purple).

FIG. 10 shows myogenic differentiation of cultured cells from 2 independent cultures (T21 and T23). The images along the left correspond to control cultures while those on the right correspond to cultures maintained in a low serum differentiation medium. Black arrows indicate myotubes.

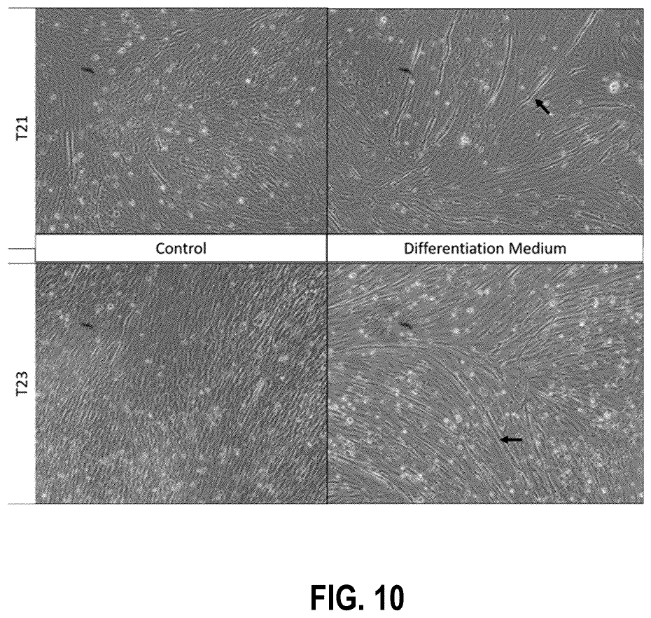

FIG. 11 shows Skeletal muscle differentiation and protein expression in fish-muscle derived cells. A) Bar graph showing BCA protein assay results which indicate average protein concentration (presented as mg of protein per mL of cells lysate) of undifferentiated and differentiated snapper cells. Cultures GS7, GS8, and GS10. B) and C) The images show the expression of sarcomeric α-actinin (B, solid white arrow) and myosin heavy chain (C, solid white arrow) in multi-nucleated myotubes in differentiated cultures. Cell nuclei are stained with DAPI (white border arrow). Scale bars are 20 μm.

FIG. 12 illustrates a flow chart for serial replating of cell cultures.

FIG. 13 shows the dependency of culture doubling time on initial seeding density.

### DETAILED DESCRIPTION

The present disclosure provides methods and processes for producing cultured meats and seafood. The cultured meats and seafood made by the methods and processes described herein are intended for use in the production of food products for human consumption.

In particular embodiments, methods and processes for producing cultured seafood is provided. As used herein, “seafood” refers to edible food obtained from a water source (such as seas, oceans, rivers or lakes) and includes both animals and plants. Categories of seafood include fish, shellfish (molluscs and crustaceans), sea cucumber, and seaweed or marine plants.

Examples of fish include, but are not limited to: anchovies, barracuda, bass, black cod, blowfish, bluefish, Bombay duck, bream, brill, butter fish, catfish, Chilean sea bass, cod, dogfish, dorade, eel, flounder, grouper, haddock, hake, halibut, herring, ilish, John Dory, lamprey, lingcod, mackerel, Mahi Mahi, monkfish, mullet, orange roughy, parrotfish, perch, pike, pilchard, ollock, pomfret, pompano, sablefish, salmon, sanddab, sardine, sea bass, shad, shark, skate, smelt, snakehead, snapper, sole, sprat, sturgeon, surimi, swordfish, tilapia, tilefish, trout, tuna (albacore tuna, yellowfish tuna, bigeye tuna, bluefin tuna, dogtooth tuna), turbot, wahoo, whitefish, whiting, or witch.

Examples of molluscs include, but are not limited to: cockle, cuttlefish, clam, loco, mussel, octopus, oyster, periwinkle, scallop, squid, conch, or nautilus. Examples of crustaceans include, but are not limited to: crab, craw fish, lobster, shrimp, or prawn.

Examples of seaweed or marine plants include, but are not limited to: algae (red algae, green algae, brown algae), arame/sea oak, badderlocks, bladderwrack, channeled wrack, carola, dulse, eucheuma, gim/kim, grapestone, hijiki, Irish moss, Carrageen moss, kelp (bull kelp, sugar kelp), kombu, nori, sea grapes, sea lettuce, gutweed, sea moss, sea palm, spiral wrack, thongweed, or wakame.

### Cell Extraction and Separation

To culture meats and seafood, tissue from an organism or from a body part of an organism is obtained and the cells are extracted. In some embodiments, the tissue is an animal tissue. In some embodiments, the tissue is a seafood animal tissue. In one embodiment, the tissue is a fish tissue. In one embodiment, the tissue is a muscle tissue.

Tissue is composed of various cells and their extracellular matrix, therefore cells are often extracted and separated before culturing. The extracted cells comprise myogenic cells, along with other cell types and debris. As used herein, “myogenic cells” refer to cells derived from muscle tissue and include muscle cells (myocytes, myotubes, myofibers) and progenitor muscle cells (such as myoblasts, satellite cells).

In some embodiments, extraction of cells involves enzymatic treatment or dissociation of the tissue. Example enzymes used in the treatment or dissociation of tissue include, but are not limited to: collagenases, peptidases (such as trypsin), lipases, or glycosidases (such as amylase).

In some embodiments, extraction and/or separation of cells involve gradient separation. In one embodiment, the gradient is in the range of about 1 to about 1.1 g/ml, preferably in the range of 1.02 to 1.08 g/mL. In one embodiment, debris is separated by density gradient separation using a separation medium. In some embodiments, density gradient separation is used to separate between different types of cells. In one embodiment, density gradient separation is used to separate between myogenic cells and fibroblasts. In one embodiment, density gradient separation is used to separate between myogenic cells and blood cells (such as red blood cells, white blood cells, or platelets).

In some embodiments, muscle cells and/or progenitor muscle cells are separated from other cells or cellular debris using one or more density gradients. Example density gradients for separating muscle cells and/or progenitor muscle cells from other cells or cellular debris include, a gradient made using a Ficoll medium, a gradient made using a Percoll medium, or a gradient made using an Optiprep™ medium. In one embodiment, two successive density gradients are used to separate muscle cells and/or progenitor muscle cells from other cells or cellular debris. In one embodiment, Ficoll-Paque separation medium is used in the separation of muscle cells and/or progenitor muscle cells from other cells or cellular debris.

In some embodiments, extraction or separation of cells include centrifugation and separation by density or mass.

### Recapture and Replating of Cell Culture

Culturing meats and seafood requires proliferation of myogenic cells. Hence the greater the number of myogenic cells extracted from tissue and captured for culture, the greater the yield. In some embodiments, the yield of muscle cells and/or progenitor muscle cells cultured from a sample cell culture is enhanced by a recapture method. In some embodiments, a sample cell culture comprising culture medium, cells extracted from a tissue, and debris are plated on a first surface (such as a dish or a vessel) to allow the muscle cells and/or progenitor muscle cells to attach. Although some of the muscle cells and/or progenitor muscle cells attach after a period of time, there are still some that remain in the culture medium unattached. The unattached muscle cells and/or progenitor muscle cells are captured by collecting at least a portion of the culture medium and replating the unattached muscle cells and/or progenitor muscle cells onto a new surface (such as a new dish or a new vessel), to allow the unattached muscle cells and/or progenitor muscle cells to attach onto the new surface. In one embodiment, the surface is a petri dish. In one embodiment, the surface is a wall (such as the side, the bottom, and/or the base) of a culture vessel. In another embodiment one or all of the surfaces are microcarrier surfaces in a bioreactor, or the internal surface of a bioreactor.

In some embodiments, extracted cells are plated on a surface coated with a substrate. In some embodiments, the substrate is laminin with or without poly-lysine, gelatin with or without poly-lysine, collagen I, collagen IV, Matrigel™, or Geltrex™. In one embodiment, the substrate is gelatin. In one embodiment, the substrate is fish gelatin.

In some embodiments, the culture medium contains fetal bovine serum (FBS) and fibroblast growth factor-2 (FGF-2). In one embodiment, the culture medium contains 15%-4% FBS by volume. In one embodiment, the culture medium contains 10%-4% FBS by volume. In one embodiment, the culture medium contains at most 70 ng/ml of FGF-2. In one embodiment, the culture medium contains at most 45 ng/ml of FGF-2. In some embodiments, the culture medium is supplemented with FGF-2 based on the FBS content. In one embodiment, the culture medium is supplemented at least 10 ng/ml FGF-2, when the FBS content of the culture medium is less than 10%.

In some embodiments, the recapture method described above is repeated again to further capture any unattached muscle cells and/or progenitor muscle cells in the culture medium of the subsequent or second plating. In some embodiments, the recapture method is repeated multiple times.

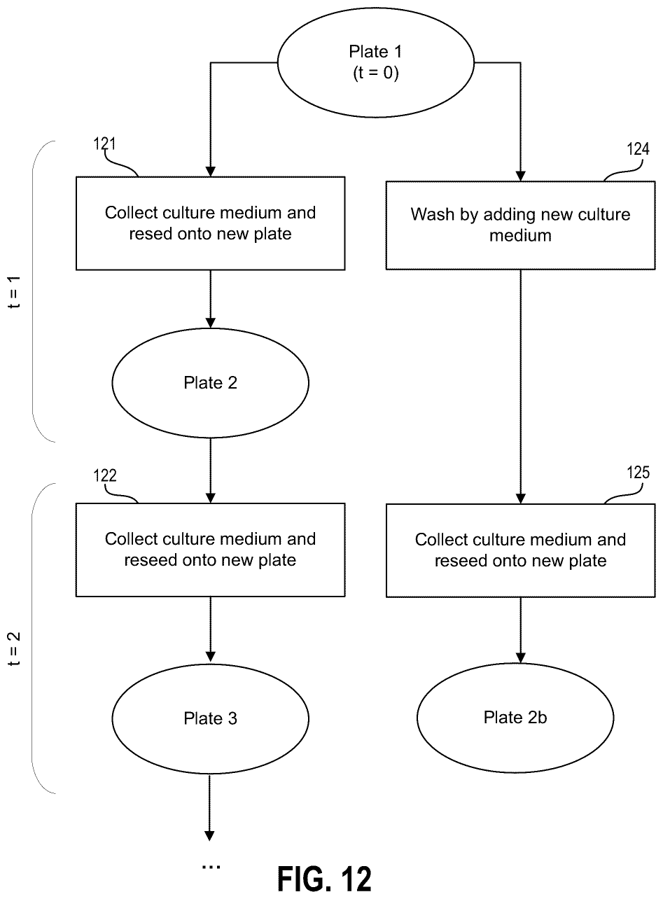

As shown in FIG. 12, Plate 1 is seeded with initial cells at t=0. up to 2 days later at t=1, the culture medium from Plate 1 is collected, which contains floating cells that did not attach to Plate 1. The collected culture medium is reused for Plate 2, thereby reseeding the floating cells onto Plate 2 (121). After a subsequent period of up to 2 days (t=2), the culture medium from Plate 2 is again collected and any floating cells are reseeded onto Plate 3 (122). This process can be repeated as needed in a series of culture medium collections and reseeding of floating cells onto a new plate, where the culture medium is recycled and reused for culturing the cells in each replating. The recycled culture medium may be supplemented with additional fresh culture medium for each replating.

Following collection of the culture medium from Plate 1, Plate 1 can also be washed by adding new culture medium (124) to collect loose cells. Att=2, the loose cells are collected by collecting the culture medium and reseeded onto Plate 2b (125). A washing step can be performed for any plate in the workflow, such as Plate 3.

By replating and/or washing, a greater number of muscle cells and/or progenitor muscle cells from the tissue sample are captured and collected for proliferation, thereby increasing the final yield.

### Cell Culture From Post-Mortem Extraction

The present disclosure provides methods and processes for culturing cells for the production of food products from organisms post-mortem. For example, cattle farming, fisheries, and food processing all invariably result in waste due to dead animals, floater fish, or scrap meats. Tissue is obtained from the dead animals or scrap meats, so that cells can be extracted and cultured for into food products. Recycling the dead animals or scrap meats in this manner allows for a greater and more sustainable source of muscle cells and/or progenitor muscle cells for culturing into food products.

In some embodiments, methods of producing a food product comprises extracting cells from a tissue sample obtained from an organism, or from a body part of the organism, post-mortem. The muscle cells and/or progenitor muscle cells isolated from the tissue sample are then cultured into a food product. In one embodiment, a tissue sample obtained from an organism, or from a body part of the organism, within 14 days post-mortem, within 10 days post-mortem, within 7 days post-mortem, within 5 days post-mortem, within 3 days post-mortem, within 2 days post-mortem, or within 1 day post-mortem.

In some embodiments, the muscle cells and/or progenitor muscle cells are cultured onto a scaffold material to support the growth of cells in three dimensions, for manufacturing into a food product. Scaffolds provide both physical support and a porous surface that facilitates the growth of cells in 3D, penetration of nutrients and the release of cell secretions. In some embodiments, scaffolds are biological scaffolds. In some embodiments, scaffolds are made from naturally occurring material.

Scaffolding materials used to produce a cultured food product must be suitable for human consumption. To be successful, the scaffold must allow for efficient growth of cells in large quantities without impacting the taste and texture of the final product. Such scaffolding materials include hydrogels based on naturally derived biopolymers such as fibrin, collagen, hyaluronic acid, alginate and chitosan. Synthetically derived polyamide and polyethene glycol polymers are also suitable as scaffolding materials. Another example scaffolding material is highly porous plant-based scaffolds, such as de-cellularized plant tissue.

In preferred embodiments, the scaffold material is collagen, hyaluronic acid, polysaccharides, or combinations thereof.

In some embodiments, culturing the progenitor muscle cells comprises culturing the isolated muscle cells and/or progenitor muscle cells with at least one other type of cells capable of secreting growth stimulatory signals. As used here, “growth stimulatory signal secreting cells” or “cells capable of secreting growth stimulatory signals” are cells that secrete substances capable of stimulating cell proliferation, healing, and/or differentiation. Growth stimulatory signals include, for example, growth factors, cytokines, and interleukins. In one embodiment, the other type of cells are allogeneic cells. Allogeneic cells are cells derived from another organism but of the same species. In other embodiments, the other type of cells are xenogeneic cells. Xenogeneic cells are cells derived from organisms of different species. In one embodiment, the at least one other type of cells are plant cells. In one embodiment, the other type of cells are autologous cells. Autologous cells are cells derived from the same organism.

In some embodiments, cells capable of secreting growth stimulatory signals are non-myogenic cells, or combination of non-myogenic cells. Example non-myogenic cells include, but are not limited to: epithelial cells, nerve cells, fat cells, bone cells, blood cells, immune cells, stem cells, pancreatic and digestive system cells, or connective tissue cells. In one embodiment, the other type of cells capable of secreting growth stimulatory signals are liver cells.

In some embodiments, the muscle cells and/or progenitor muscle cells and the growth stimulatory signal secreting cells are cultured together. In one embodiment, the muscle cells and/or progenitor muscle cells and the growth stimulatory signal secreting cells are co-cultured while separated by a microporous membrane. The microporous membrane allows the growth stimulatory signals to diffuse through to the muscle cells and/or progenitor muscle cells, thereby promoting proliferation of the muscle cells and/or progenitor muscle cells. In some embodiments, the muscle cells and/or progenitor muscle cells are cultured separately from the growth stimulatory signal secreting cells. In one embodiment, the culture medium from a culture containing the growth stimulatory signal secreting cells is collected and introduced to a culture containing muscle cells and/or progenitor muscle cells. In this manner, growth stimulatory signals secreted by the growth stimulatory signal secreting cells into the culture medium are collected and introduced to the muscle cells and/or progenitor muscle cells, thereby promoting proliferation of the muscle cells and/or progenitor muscle cells.

### Food Products and Other Applications

In some embodiments, the cultured muscle cells and/or progenitor muscle cells described herein are intended for making into food products. In accordance with the present disclosure, the cultured muscle cells and/or progenitor muscle cells produced by the methods and processes described herein are further prepared into food products.

Example food products that can be prepared from cultured seafood include, but are not limited to: fish fillets, smoked fish, fish flakes, crab meat, breaded or battered seafood, fish sticks, fish ball, fish nuggets, fish patties, sashimi, squid rings, surimi or minced seafood.

In some embodiments, a food product made by the methods described herein contains fish muscle cells and optionally at least one other seafood cell. In one embodiment, a food product contains one or more different fish muscle cells. In one embodiment, a food product contains muscle cells from one or more different species of fish. In one embodiment, a food product contains muscle cells from two different species of fish. In some embodiments, a food product comprises: a) cultured fish muscle cells, either alone or in combination with: b) other cultured cells, c) animal derived ingredients and/or d) non-animal derived ingredients. In one embodiment, a food product comprises first cultured fish muscle cells. In one embodiment, a food product comprises first cultured fish muscle cells and seafood cells. In one embodiment, a food product comprises first cultured fish muscle cells and other cultured cells. In one embodiment, a food product comprises first cultured fish muscle cells and second cultured fish muscle cells. In one embodiment, a food product comprises cultured fish muscle cells and cultured or non-cultured seafood cells. In some embodiments, the food product further comprises animal derived ingredients and/or non-animal derived ingredients.

In some embodiments, a food product contains muscle cells from one or more different fish and a plant seafood cell. In one embodiment, a food product contains cultured fish muscle cells and algae cells.

In some embodiments, the cultured muscle cells and/or progenitor muscle cells described herein are intended for producing normal or diseased tissue models. The tissue models can be used for research or diagnostics. In some embodiments, the cultured muscle cells and/or progenitor muscle cells described herein are intended for therapeutic uses, such as tissue replacement therapy or drug testing and assays.

The present invention will be more readily understood by referring to the following examples which are given to illustrate the invention rather than to limit its scope.

### Examples

The following examples illustrate certain embodiments addressing specific design requirements and are not intended to limit the embodiments described elsewhere in this disclosure.

### Culture Medium

Media used in the process have been designed to minimize use of materials that may not be suitable for use in manufacture of a food product. Media do not include phenol red, commonly present as a pH indicator in culture media, but not suitable for food. In addition, animal-derived medium components from non-Teleost species have been minimized (all media will be further optimized).

Isolation medium—Hank's Balanced Salt Solution (HBSS), without phenol red, but with a 2× concentration of antibiotic/antimycotic (200 units/mL of penicillin, 200 μg/mL of streptomycin, and 500 ng/ml of Amphotericin B).

Wash medium—Leibovitz's L-15 Medium, without phenol red, but with a 2×concentration of antibiotic/antimycotic (200 units/mL of penicillin, 200 μg/mL of streptomycin, and 500 ng/ml of Amphotericin B).

Seeding Medium—Leibovitz's L-15 Medium, without phenol red, is supplemented with 15% fetal bovine serum (FBS) or alternatively a number of non-animal-derived growth factors and other serum supplements. These supplements are from algae (Spirulina extract, BYAS CA101, Corbion products, Algenuity products, Algatech products), plants (Hy-Soy, HyPeP 1510, Hy-Pep 7504, Hy-Pep 4601, UltraPep Soy) or yeast (Yeastolate), or are proprietary mixtures (Ultroser-G, Sericin, Knock-Out Serum Replacement, B27 supplement). In addition, other supplements include insulin (10 μg/mL), transferrin (5.5 μg/mL), sodium selenite (6.7 ng/ml), lipids and amino acids. The medium also contains a 2× concentration of antibiotic/antimycotic (200 units/mL of penicillin, 200 μg/mL of streptomycin, and 500 ng/ml of Amphotericin B).

Maintenance Medium—Leibovitz's L-15 Medium, without phenol red, is supplemented with 5-10% fetal bovine serum (FBS) or alternatively a number of non-animal-derived supplements. These supplements are from algae (Spirulina extract, BYAS CA101, Corbion products, Algenuity products, Algatech products), plants (Hy-Soy, HyPeP 1510, Hy-Pep 7504, Hy-Pep 4601, UltraPep Soy) or yeast (Yeastolate), or are proprietary mixtures (Ultroser-G, Sericin, Knock-Out Serum Replacement, B27 supplement). In addition, other supplements include insulin (10 μg/mL), transferrin (5.5 μg/mL), sodium selenite (6.7 ng/ml), lipids and amino acids. The medium also contains cell growth signals in the form of growth factors, including fibroblast growth factor-2 (FGF-2; 0-70 ng/ml), dexamethasone (10-25 nM) (Larson et al., 2018; Lagendorf et al., 2020) insulin-like growth factor-1 (IGF-1; 25-100 ng/ml) (Engert et al., 1996; Milasincic et al., 1996; Bower & Johnston, 2010; Yu et al., 2015), ascorbic acid (200 UM) (Duran et al., 2019), and thyroxine (5-50 ng/ml) (Milanesi et al., 2016; Lee et al., 2017) 2. The medium also may contain antibiotics and antimycotics which are at a 1-2× concentration at the initiation of primary culture (100-200 units/mL of penicillin, 100-200 μg/mL of streptomycin, and 250-500 ng/ml of Amphotericin B). This mixture is reduced by half at the first passage and is removed from later passages.

Differentiation Medium—Leibovitz's L-15 Medium and/or Dulbecco's Modified Eagle Medium (DMEM), without phenol red, is supplemented with 2% fetal bovine serum (FBS) or alternatively a number of non-animal-derived supplements. These supplements are from algae (BYAS CA101, Corbion products, Algenuity products, Algatech products), plants (Hy-Soy, HyPeP 1510, Hy-Pep 7504, Hy-Pep 4601, UltraPep Soy) or yeast (Yeastolate), or are proprietary mixtures (Ultroser-G, Sericin, Knock-Out Serum Replacement, B27 supplement). In addition, other supplements include insulin (10 μg/mL), transferrin (5.5 μg/mL), sodium selenite (6.7 ng/ml), lipids and amino acids (1% Essentials and Non-essentials amino acids solutions). The medium also contains factors that stimulate myogenic differentiation, including dexamethasone (1-10 UM) (Han et al., 2017; Larson et al., 2018; Lagendorf et al., 2020), insulin (10 μg/mL) (Afshar et al., 2020), IGF-1 (25-500 ng/mL) alone or in combination with amino acids (Miyata et al. 2017; Vélez et al. 2014; Gabillard et al. 2010; Engert et al., 1996), and thyroxine (5-50 ng/ml) (Milanesi et al., 2016; Lee et al., 2017).

### Post-Mortem Cell Isolation Process

The isolation process involves sequential mechanical and enzymatic dissociation and was initially based on published protocols (Koumans et al., 1990; Fauconneau and Paboeuf, 2000; Montserra et a., 2007; Froehlich et al., 2014). Modifications were implemented to reduce washing steps as a means of minimizing cell loss, as well as removal of animal derived components from non-Teleost species, such as removal of horse serum, and the replacement of trypsin with non-animal-derived TrypLE (ThermoFisher™).

Source muscle tissue is obtained from lean white fish (in the example here Nile Tilapia, Oreochromis niloticus; Gray Snapper, Lutjanus griseus; and Largemouth Bass, Micropterus salmoides) was the species used, either immediately after euthanasia or after many days post-mortem. Other lean white fish include, but are not limited to, Atlantic halibut (Hippoglossus hippoglossus), Haddock (Melanogrammus aeglefinus), Sablefish (Anopoploma fimbria), Atlantic cod (Gadus morhua), sole (various species), sea bass (various species), snapper (various species).

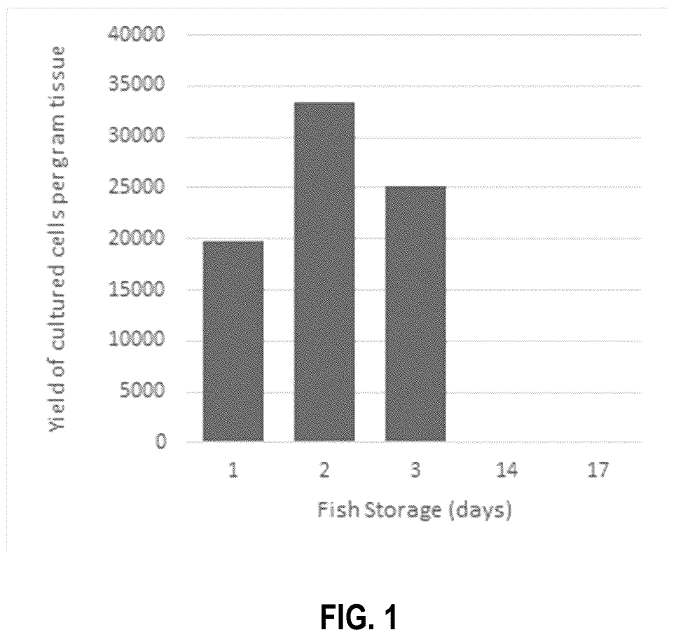

For post-mortem isolation, the whole fish is stored at 2-8° C. in the refrigerator until processed. FIG. 1 shows data on cell culture yield after isolation from intact fish stored at refrigerated temperatures for 1 to 17 days, with the data normalized to the amount of tissue processed. Large numbers of cells could be grown from fish dead for 1-5 days, whereas no viable cell cultures were generated using fish dead for 2 weeks. The present inventors have discovered the finding of effective culture from tissue many days after death in a Teleost species. Prior studies describe post-mortem isolation of muscle stem cells in human and mouse muscle and isolation of other types of non-muscle cells (Latil et al., 2012; Mansilla et al., 2013).

The fish undergo surface decontamination to prevent microbial or fungal contamination of muscle cell cultures. The fish is first rinsed in fresh water to remove mucus and skin scales are removed by scraping with a hard-edged implement (e.g. closed scissors). Then the fish is transferred to a solution of 0.05% bleach and the surface rubbed and gill areas rinsed for 5 minutes at room temperature. The fish is then soaked in 70% isopropanol and rubbed for another 5 minutes at room temperature.

The fish is transferred into a cell culture hood (laminar flow cabinet or biological safety cabinet) for dissection. One side of the fish is dissected at a time. An incision is made close to the dorsal fin and runs along the length of the body. Another incision is perpendicular and runs from the top of the fish and proceeds ventrally just behind the operculum. The skin is then carefully dissected away to expose the muscle. Muscle progenitors have been isolated from epaxial muscle (from just above midline to top of fish), hypaxial muscle (just below midline to abdominal cavity), and from the red muscle at the midline. Muscle tissue is dissected away from ribs and vertebral processes and placed in a Petri dish, and the amount of tissue weighed.

The muscle tissue first undergoes mechanical dissociation and processes have been developed based on the amount of tissue to be processed. For small scale processing (<100 g tissue), 5-gram portions are processed individually, whereas with large scale processing (100 g and above), the tissue is processed using multiple operators working with a proportional amount of the total tissue (e.g. one half for each of two operators). The tissue is manually minced to cut down into small pieces (approximately <2 mm³ and flattened) through use of dissection scissors and scalpels. Once minced, the tissue is transferred to a container with Isolation medium. For small scale process, 5 grams of minced tissue is transferred into individual tubes containing 24 mL of Isolation medium, whereas larger amounts of tissue will scale-up the amount of Isolation medium, so it is proportional to small-scale.

A two-part enzymatic dissociation process is described that extracts cells from minced muscle tissue and is first treated with collagenase followed by a trypsin-like enzyme of non-animal origin. Prior studies for enzymatic isolation of muscle cells from Teleost species have utilized a variety of approaches, including use of collagenase alone or coupled with another enzyme (trypsin, neutral protease/dispase). Of particular relevance are those methods utilizing trypsin, however, this enzyme is animal-derived (porcine) (Powell et al., 1989; Koumans et al., 1990). In the process described here collagenase (e.g. Gibco type I collagenase, non-animal origin) is added to the Isolation medium containing tissue, such that a concentration of 2 mg/ml is achieved. The collagenase:tissue solution is incubated for 1 hour at 27° C. with strong agitation. The tissue fragments are then triturated, with ten passes using a 10 mL pipette for large-scale preparations, while for small-scale this is followed by trituration through a 5 mL pipette and then passage through a 16-gauge needle. The collagenase breaks down extracellular collagen, however, further digestion with a trypsin analogue of non-animal-origin (e.g. Gibco TrypLE) is performed to increase cell extraction. For small-scale isolations, 10 mL of trypsin analogue is added to each tube containing 5 grams tissue, whereas with large scale isolations the equivalent volume:tissue is maintained in a larger vessel. Incubation with trypsin analogue is 20 minutes at 27° C. with strong agitation.

After completion of one round of trypsin analogue incubation, the enzyme digestate is filtered and remaining tissue fragments undergo a second round of trypsin analogue incubation. For a large-scale isolation, the solution is filtered through a 255 μm strainer to capture tissue for the second trypsin analogue incubation. Both isolation scales undergo sequential filtering through a 105/100 μm strainer (large and small scale, respectively), followed by a 40 μm strainer. Enzyme activity is quenched by addition of protein-rich medium containing serum or a solution of a protein such as albumin. The filtered suspension is kept cold (on ice or refrigerated). The suspension from the second round of trypsin analogue is processed in the same way as the first, except for omission of the 255 μm filter.

The filtered enzyme digestates undergo volume reduction by centrifugation (400-500×g, 12° C., 10-20 minutes) and resuspension in a 2.5-fold smaller volume of HBSS without calcium or magnesium ions to prevent adherence to the tube or cell aggregation.

Tissue debris is removed from the cell suspension by density gradient separation using a medium of 1.077 g/mL density (e.g. Ficoll-Paque PLUS, Cytiva). Previous studies with Ficoll density gradient separation has focused primarily on isolation of specific cell populations from various tissues (such as blood, sperm, etc.) (Boyum, 1968; Pretlow & Pretlow, 1977). This method has also been used to purify a tissue preparation by removing contaminating bacteria (Attree & Sheffield, 1986), including Teleost muscle (Araki, 2009; Alexander, 2011). Here, Ficoll is primarily used to separate tissue debris from the isolated cells prior to initiation of cell culture.

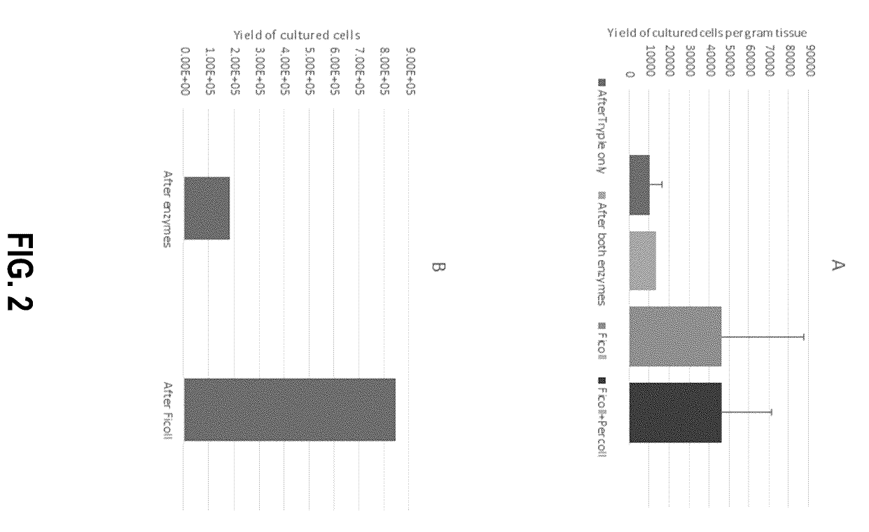

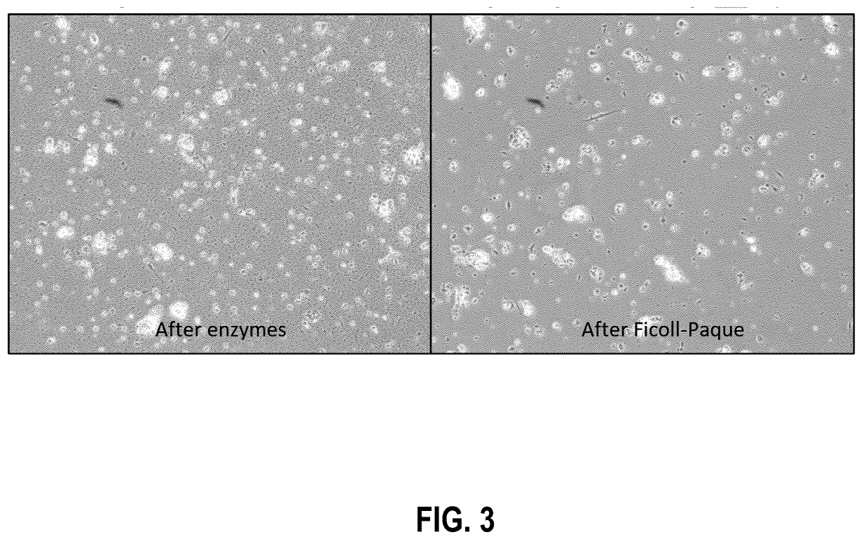

The process described here involves slowly layering a 25 mL volume of concentrated tissue digest on top of 20 mL of Ficoll-Paque PLUS at 1.077 g/mL, within a 50 ml tube. The tubes are then centrifuged at 1400×g at 9-20° C. for 40 minutes, with gradual acceleration and no brake. After centrifugation, a number of fractions can then be discerned, including an upper HBSS layer, a lower layer of Ficoll-Paque, an interface between layers where the bulk of cells will accumulate, and a pellet composed mainly of tissue debris and erythrocytes. The HBSS layer, interface, and a small portion of Ficoll-Paque (approximately 5 mL) are collected and may be pooled together. Implementation of density gradient separation of debris has resulted in a large increase in subsequent cell culture yield compared to seeding cultures immediately after TrypLE, with or without the cells released by collagenase (FIG. 2, data normalized to amount of tissue processed). FIG. 3 illustrates the reduction in debris evident early in cell cultures when initiated directed after enzyme treatment compared to when initiated after Ficoll-Paque density gradient separation.

Following debris removal, the isolated cell suspension is further refined to isolate myogenic stem cells and progenitors (also known as satellite cells and myoblasts, respectively) from connective tissue fibroblasts. To achieve this, density gradient separation using a variable gradient of the range of 1.02-1.08 g/mL, either as a continuous or discontinuous gradient. In one embodiment, Percoll (Cytiva) is diluted with HBSS to prepare the required densities, which would be in the range of 15% to 70% Percoll. To prepare a discontinuous gradient, immediately before use, individual layers with different densities are added to a tube with the densest added first to the bottom, and then each subsequent layer added carefully with decreasing density. A continuous gradient can be prepared, for example, by adding a Percoll solution in the middle of the required density range to a tube and centrifuged at 17,000×g for 15 minutes at 20° C. To process the cell suspension, it is first carefully layered on top of the Percoll gradient and centrifuged at 1800×g for 60 minutes at 20° C., with gradual acceleration and no brake. To assess the efficacy of the separation, density marker beads of a suitable density range are loaded onto a separate gradient tube and centrifuged alongside the cell suspension. The components of the cell suspension will resolve into layers that can be collected using a Pasteur pipette and used to initiate cell cultures. Sequential density gradient separation through Ficoll-Paque and Percoll was found to have minimal impact in the number of cells that could be grown in culture (FIG. 2). Prior studies have demonstrated that Percoll gradients can separate subpopulations of skeletal muscle cells in avian (Yablonka-Reuvneni et al., 1987; Yablonka-Reuveni & Nameroff, 1987) and mammalian species (Morgan, 1988; Bischoff & Heintz, 1994). However, Greenlee et al. (1995) found that application of a Percoll gradient did not enrich cultures for Trout myogenic cells. Percoll has also been used in preparing cultures from muscle of Channel catfish, however, in this application the Percoll was added temporarily to the culture and not with centrifugation (Mulvaney & Cyrino, 1995). Cultures of myogenic and fibroblastic cells were generated from Tilapia cell suspensions after separation on Percoll gradients. Other cell types could be isolated and removed, such as red blood cells, which were found at the interface between 40% and 70% Percoll layers, and cells with macrophage-like morphology, which were found in the 25% Percoll layer. These layers contained negligible myogenic/fibroblastic cells. Continuous Percoll gradients can be used to potentially separate between myoblasts and fibroblasts and thus enrich for a pure(er) population of myoblasts to be cultured. The ability to segregate myoblast and fibroblast populations is ideal for manipulation of the relative proportions of these cells in culture such that a population optimized for a cultured meat product is achieved.

Following density gradient separation, the pooled cell suspension is then washed and concentrated. The suspension is centrifuged (400-500×g, 12° C., 10-20 minutes) and the pellet resuspended in Wash medium, centrifuged again, and resuspended in Seeding medium. The volume of Seeding medium is dependent on the amount of tissue processed and the size of culture vessel used. For 50 grams of tissue, a culture of approximately 20 cm² surface area will be initiated, while 100 grams could initiate 50-75 cm². The surface of the culture vessels is coated with laminin to facilitate cell adhesion. The volume of Seeding medium would be 1 mL per well of a 6-well plate (approximately 10 cm²), 3 mL for a T25 flask (25 cm²), and 10 mL for a T75 flask (75 cm²). The culture vessels are coated with substrate prior to use (laminin with or without poly-lysine, gelatin with or without poly-lysine, collagen I, collagen IV, Matrigel, Geltrex). Of particular interest for seafood produced from cells is the use of fish gelatin as a substrate for cell culture.

### Serial Recapture Method

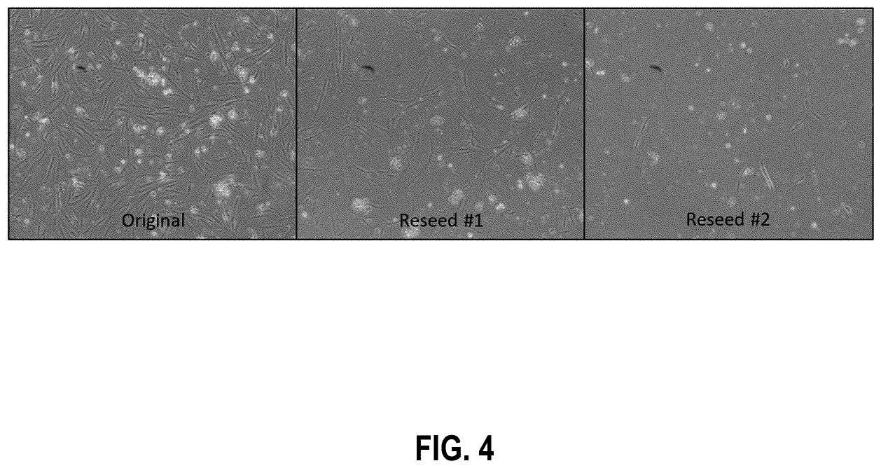

The cell cultures undergo medium changes over the first two days as a means to i) remove any residual tissue debris, ii) refresh the culture medium, and iii) capture any cells that have not become adherent yet by reseeding into a fresh substrate-coated culture vessel (i.e. recycling of old medium) as described above. Removal of debris and refreshing the medium are standard practices, however, the inventors have presently developed a technique of serial recapture of isolated cells by collecting and recycling the culture medium (containing cells) used in muscle cell culture. Prior studies of muscle cell culture have utilized a different technique (preplating) as a means to segregate fibroblasts from myogenic cells (“preplating”) and not increase cell attachment (Yaffe, 1968). Preplating has also been applied to Teleost species (Koumans et al., 1990; Alexander et al., 2011). It is possible that cultures of mixed cell types may confer an advantage related to soluble stimulatory factors secreted by a subpopulation of cells. In the technique described here, up to 2 days after initiating cell culture (t=1 in FIG. 12), the culture medium is transferred to a fresh vessel of the same size (Reseed #1, “Plate 2” in FIG. 12) and the original culture receives fresh culture medium. After a subsequent period of up to 2 days (t=2 in FIG. 12), the medium from the original culture is transferred to a fresh vessel of the same size (Reseed #2, “Plate 2b” in FIG. 12). The recycled cultures are treated in the same way as the original cultures with the initial feeding being either Seeding medium or Maintenance medium (containing FGF-2). The culture medium of all cultures is changed every 1-10 days thereafter, depending on cell density, with replacement of either half or full volume of fresh Maintenance medium. Cell growth has been demonstrated in reseeded/recaptured cultures (FIG. 4). Additional reseeding/recapture procedures can be done with media removed from the primary culture and the recycled cultures (later washes).

### Co-Culture Systems

An alternative culture system is to culture isolated muscle cells with factors secreted by another source of cells and these paracrine secretions can provide growth stimulatory signals to enhance cell proliferation. The source of these signals would be cells derived from various tissues, and not limited to muscle tissues. For example, cells from seafood tissue, plant tissues, algae, fungi tissue, or other animal tissue can be the source of these signals. As well, cells from tissues such as gills, scales, bones, liver, and neurological tissue can be used as sources of these signals. Although defined growth factors are known to originate from certain cell types, the essence of the claim is that it is the mixture of signals produced, be it a single or multiple cell types present in a tissue, that offers a better growth stimulatory signal. This is achieved in multiple ways, with one method to culture both muscle cells and signal-producing cells in an indirect co-culture system, whereby the different cell populations are separated by a microporous membrane. Exemplary co-culture systems are described in US20190376026A1, the entire contents of which are incorporated herein by reference. The pores of the membrane are of a sufficiently small size to prevent cell migration (e.g. 0.4-1 μm) but will not impede soluble signaling molecules. Alternatively, the signal-producing cells may be cultured separately, and the soluble signals collected by harvesting the conditioned culture medium and applying that to the muscle cell culture. Although prior studies have used such methods in other cellular contexts, the present inventor has developed this method for stimulation of fish muscle cell growth through complex cellular secretions.

### Culturing Seafood

Cell cultures are maintained until density reaches approximately 70-100% confluence, at which point the cells are detached and used to initiate new cultures to continue cell proliferation, and thus expand overall cell number. The culture medium is removed from the culture vessel, which is then washed to remove residual medium (e.g. Dulbecco's phosphate-buffered saline without calcium or magnesium ions). One method to detach cells utilizes a non-animal-derived trypsin-like enzyme (e.g. Gibco TrypLE), which is applied on the cells, which are observed under a microscope and removed when significant cell detachment is detected. Multiple cycles of enzyme and repeated pipetting and scraping may also be used to dislodge cells. An alternative technique is to treat the cells with an EDTA solution (e.g. 0.5 mM EDTA) for approximately 5-10 minutes and then detach the cells using a scraper. After either method, the cell suspension is diluted with culture medium to neutralize the dissociation agent and a cell count is performed. The cell suspension is centrifuged (400-500×g, 15 minutes) and the pellet resuspended in Maintenance medium prior to seeding in a culture vessel coated with substrate prior to use (laminin with/without poly-lysine, gelatin with/without poly-lysine, collagen I, collagen IV, Matrigel, Geltrex).

A variation of the culture process is to combine different batches of cells, be they of the same species or of different species. It is anticipated that cultured muscle cells will not be identical between individual fish of the same or different species and thus it would be possible to create a mixed cell population based on desired criteria relevant to cellular agriculture (e.g. muscle fiber size, fat content, flavour, structure, etc.). As the muscle cell cultures do not contain significant numbers of immune cells, there is no reason to believe that there would be any immune reaction between the cells from different individuals/species. An example situation would be augmenting the taste profile of Tilapia by including the firmness offered by Halibut through mixing together 33% Tilapia cells with 67% Halibut. It is possible that the isolation procedure for different species may require minor adjustment, however, it is unlikely that the culture medium would need to be customized and therefore a mixed cell culture is anticipated to be maintained with the standard methods.

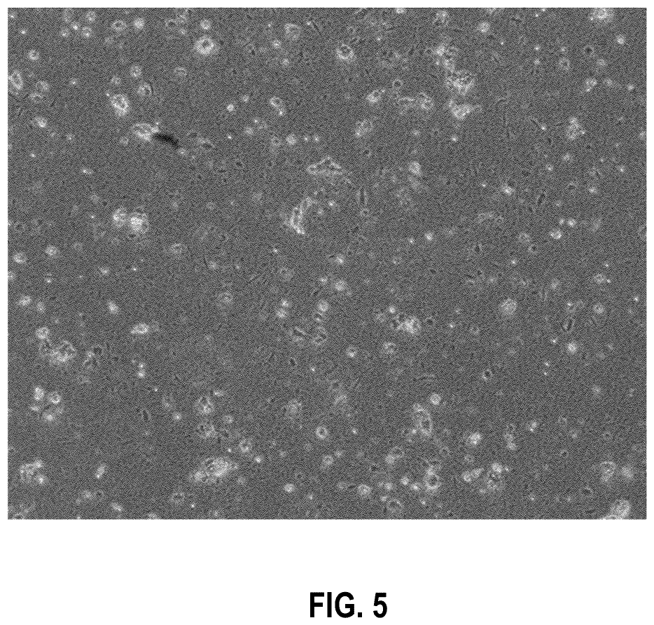

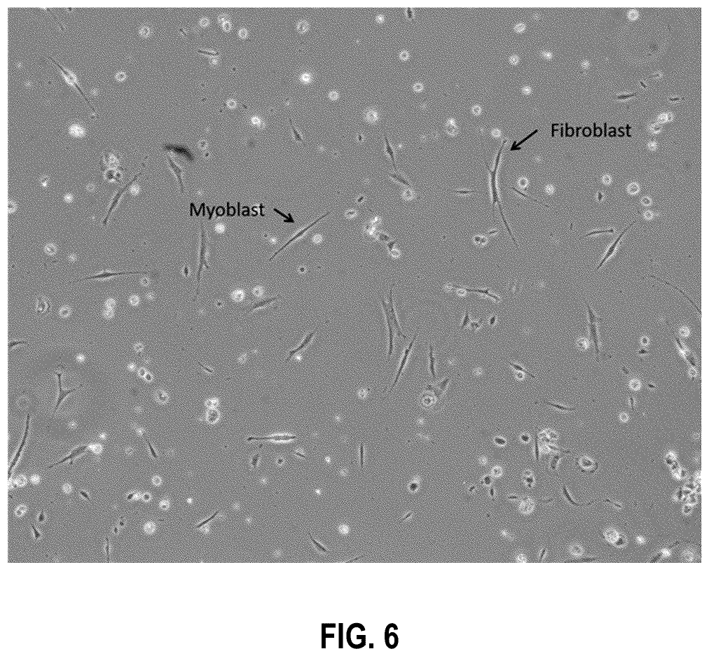

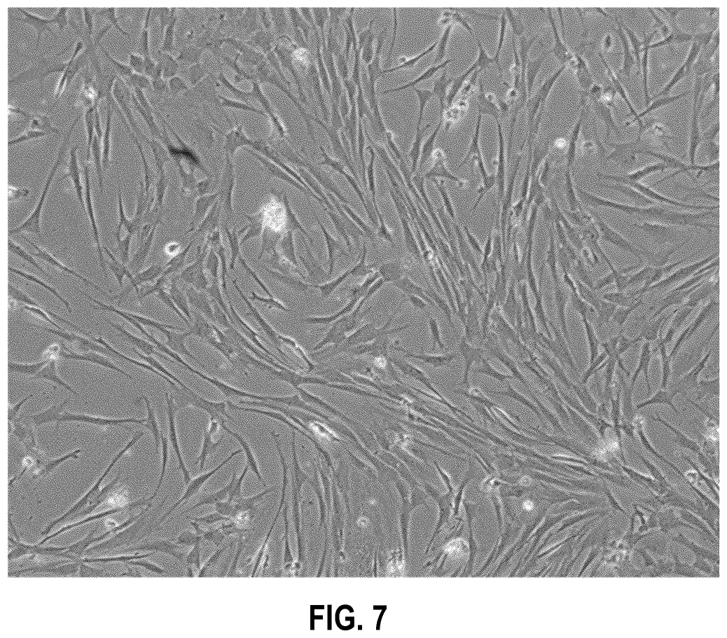

The kinetics of cell growth in the primary cultures is reproducible. In the first days of culture, the cells have a rounded shape and there is residual debris and aggregates from the isolation, which will be removed with subsequent medium changes (FIG. 5). Within a few days of culture, the cells take on distinctive morphologies of myoblasts and fibroblasts (FIG. 6), and the cells subsequently proliferate, and the cell layer becomes confluent within 1-2 weeks (FIG. 7). As described above, when the cultures reach 70-100% confluence, they are passaged into new culture vessels.

Cell lines derived from fish muscle have been previously reported, however, the vast majority of these are fibroblastic in nature and not capable of myogenic differentiation (Middlebrooks et al., 1979; Hedrick et al., 1991; Fernandez et al., 1993; CN104004707A). Gignac et al. (2014) has reported derivation of a continuous cell line with myogenic characteristics derived from non-commercial Mummichog (Fundulus heteroclitus). The work described here with cell lines from commercially relevant species demonstrate cell proliferation over extended periods of time and over many passages resulting in high numbers of population doublings (e.g. one subline of GS7 has been cultured for 260 days over 72 passages and has reached a population doubling level (PDL) of 79), with a doubling time as low as 16 hours, which is significantly lower than the 3 days that we could find in the fish scientific literature (Gignac et al., 2014).

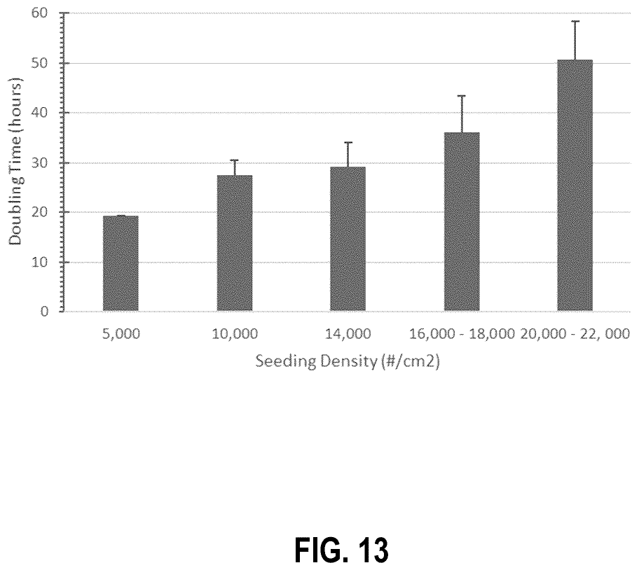

The density of cultures when initiated (i.e., seeding density) was found to influence their proliferative potential. Experiments with cell line GS7 were performed with a seeding density of 5,000-22,000 cells/cm² (see table below) and culture conditions of laminin substrate, 15% FBS, and 15 ng/ml FGF, and the cultures harvested after 3 days. The doubling time was calculated as a measure of cell proliferation and is the time taken to double the number of cells. As the seeding density increased, the doubling time also increased (see figure below). Presumably as the cells replicated and culture density increased, the cells became contact inhibited and cell proliferation decreased, resulting in a longer doubling time. At the lowest seeding density, the cultures exhibit maximal proliferation and the lowest doubling time (19.2 hours with 5,000/cm² seeding). For comparison, the myogenic fish cell line described by Gignac et al. (2014) was described as having a doubling time within 3 days. (See Table 1 and FIG. 13)

TABLE 1
Dependency of culture doubling time on seeding density

| | Doubling Time |
|---|---|
| | (hours, average ± standard |
| Seeding Density | deviation, with number of |
| (number per cm²) | experiments in parentheses) |
| 5,000 | 19.2 ± 0.05 (3) |
| 10,000 | 27.5 ± 3.1 (3) |
| 14,000 | 29.2 ± 4.9 (3) |
| 16,000-18,000 | 36.0 ± 7.4 (4) |
| 20,000-22,000 | 50.6 ± 7.7 (7) |

A number of parameters have been assessed as to their impact on cell proliferation. Suitable incubation temperature for the cultures was in the range of 89-133% of the habitat temperature for the species. Variation in mitogenic components of the culture medium, FBS (4-15%) and FGF-2 (0-70 ng/ml), were also assessed. Reduction of FBS concentration, as would be appropriate for a cell-based seafood product, can also result in lower cell proliferation (Gignac et al., 2014), however, this can be compensated for by increasing FGF-2 levels (see Table 2), with cell proliferation assessed by expansion in number over a culture passage (i.e. output/input×100%). This is consistent with the known function of FGF as a mitogen for mammalian myoblasts (Gospodarowicz and Mescher, 1977).

TABLE 2
Effect of fetal bovine serum (FBS) and FGF-2 content
in culture medium on cell expansion in GS8 cell line

| FBS | FGF-2 | Number of | Average Cell Expansion |
|---|---|---|---|
| (volume %) | (ng/mL) | cultures | (range) |
| 15% | 5 | 8 | 290% (236-333%) |
| 10% | 5 | 4 | 270% (171-378%) |
| 9% | 5 | 7 | 167% (82-220%) |
| 9% | 10 | 6 | 124% (69-203%) |
| 8% | 15 | 7 | 169% (80-270%) |
| 7% | 15 | 4 | 176% (129-202%) |
| 6% | 15 | 3 | 199% (181-215%) |
| 5% | 15 | 8 | 166% (123-270%) |

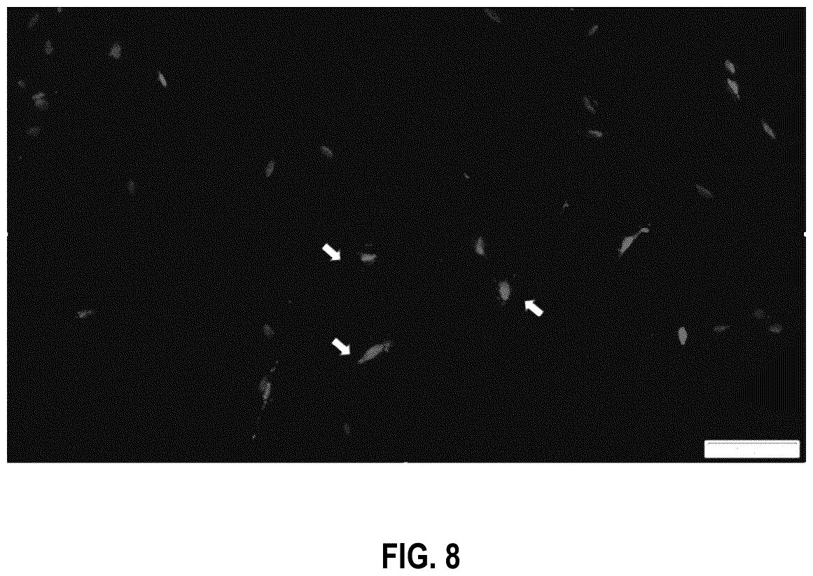

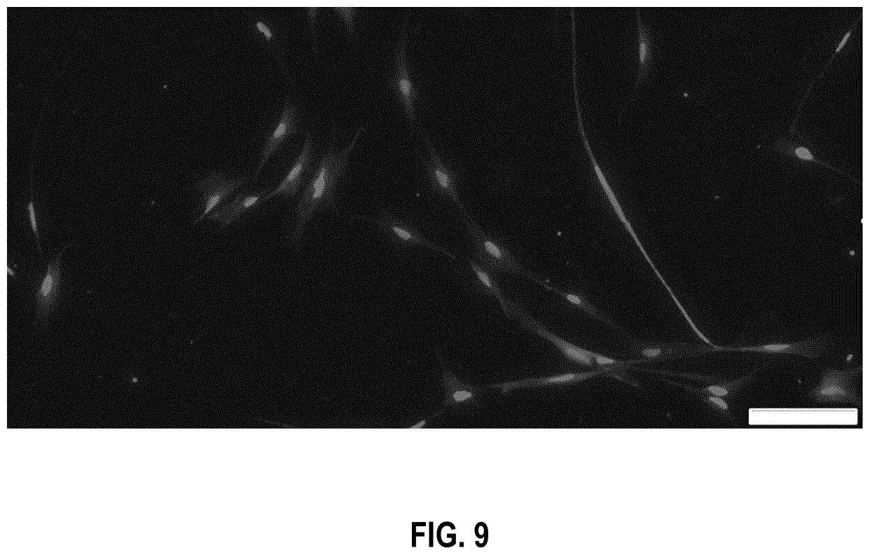

The cultured cells express markers consistent with early muscle cells by immunofluorescence staining. The cells stain positively for Pax3 (FIG. 8), a transcription factor that is expressed in muscle progenitor cells, and desmin (FIG. 9), which is a cytoskeletal protein found in early muscle cells muscle progenitors and differentiated muscle cells.

### Muscle Cell Differentiation

Skeletal muscle cells can be differentiated into more mature muscle cell types, multi-nucleated myotubes. In general, in muscle cell cultures, including the ones from teleost fish, spontaneous differentiation can be achieved by maintaining the cells highly confluent for an extended period of time (Millan-Cubillo et. Al., 2019; Duran et al., 2015; Froehlich et. al., 2014; Gabillard et al., 2010). Differentiation can be further enhanced by using differentiation media containing a lower concentration of animal serum (e.g., fetal bovine serum or horse serum) with or without further supplementation of myogenic stimulating factors (e.g., IGF-1 and Insulin, transferrin) (Kong et. al., 2021; Afshar et. Al., 2020; Miyata et. al., 2017; Alexander et. al 2011; US20080113435; WO2020149791). We first followed the same procedure to directly induce differentiation by seeding muscle-derived cells in laminin/poly-lysine coated tissue culture plates (at a seeding density of 22,000 to 27,000 cells/cm²). Then, upon reaching ≥90% confluence, the maintenance medium was replaced with differentiation medium consisting of L-15 supplemented with 2% FBS, 1 UM insulin, 100 ng/ml IGF-1 to induce myoblast differentiation for up to 10 days. Myogenic progression to multi-nucleated myotubes was evident as early as differentiation day 2. However, we observed a gradual reduction in the quality of the cultures as indicated by the presence of extensive cytoplasmic vacuolization in myotubes and cell loss in the late stages of differentiation (days 5-10).

A two-step differentiation protocol was developed combined with reduction of incubation temperature to improve the quality of cultures with higher protein content, which is an important parameter for a food product. Muscle-derived cells were seeded in laminin/poly-lysine coated tissue culture plates at the same seeding density (22,000 to 27,000 cell per cm²). Upon reaching ≥90% confluence the maintenance medium was exchanged with FGF-2-free maintenance medium (L15 supplemented with FBS) and the incubation temperature was decreased to 25° C. (pre-differentiation step). It was hypothesized that, before inducing differentiation, maintaining the cells at high confluence with reduced mitogen-containing culture medium and lower temperature would prime the cells for improved differentiation. Following up to 5 days pre-differentiation, culture medium was exchanged with the same differentiation medium (L-15 supplemented with 2% FBS, 1 μM insulin, 100 ng/ml IGF-1) to further induce myogenic differentiation. The muscle differentiation samples were maintained in differentiation medium for up to 5 days at 25° C.

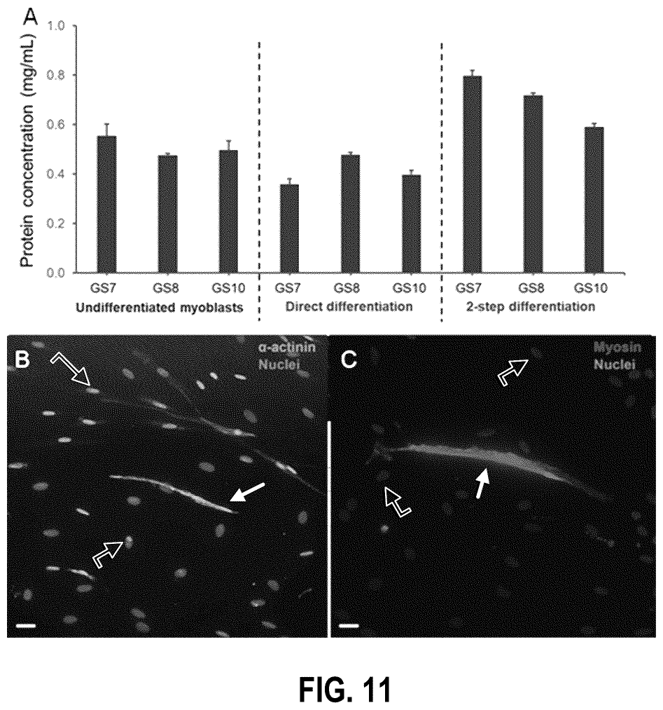

At the end of the pre-differentiation step, spontaneous differentiation was observed as indicated by the presence of a few small multi-nucleated myotubes. Although a few myotubes with cytoplasmic vacuolization were seen, the extent of vacuolization and cell loss was much lower compared to that seen during direct differentiation. Therefore, pre-differentiation can smoothen the transition to the differentiation stage and increase the yield of differentiation. At the late differentiation period, we observed formation of well-developed multi-nucleated myotubes without severe cell loss. This indicates that the two-step differentiation protocol along with reduced culture temperature can substantially improve the outcome of the cultures as negligible detachment of cells and presence of multiple well-developed myotubes were observed. In line with morphological assessments, the protein assay showed that the protein content in two-step differentiation conditions (evaluated in GS7, GS8, and GS10 lines) was significantly higher than the direct differentiation cultures and undifferentiated myoblasts (FIG. 11, panel A, and Table 3 below).

TABLE 3
Protein content from muscle cell lines differentiated
under different conditions

| Differentiation | Muscle-derived | Protein concentration | |
|---|---|---|---|
| condition | cell line | (mg/mL) | SD |
| Undifferentiated | GS7 | 0.55 | 0.05 |
| myoblasts | GS8 | 0.48 | 0.01 |
| | GS10 | 0.50 | 0.04 |
| Direct | GS7 | 0.36 | 0.02 |
| differentiation | GS8 | 0.48 | 0.01 |
| | GS10 | 0.40 | 0.02 |
| Two-step | GS7 | 0.80 | 0.02 |
| differentiation | GS8 | 0.72 | 0.01 |
| | GS10 | 0.59 | 0.01 |

Furthermore, multi-nucleated myotubes were found to express sarcomeric α-actinin (FIG. 11, panel B) and myosin heavy chain (FIG. 11, panel C) which are indicators for maturation of muscle cells. Taken together, these experiments confirm that a notable number of myogenic progenitors retain the capacity to differentiate into mature myotubes in our cell culture system regardless of the number of sequential passages. Furthermore, the two-step differentiation protocol at lower incubation temperature appears to provide superior differentiation conditions for fish muscle-derived cells.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention. All documents and publications referenced herein, including but not limited to those in the following reference list, are incorporated by reference in their entirety.

### REFERENCES

1. Afshar, M. E., Abraha, H. Y., Bakooshli, M. A. et al. A 96-well culture platform enables longitudinal analyses of engineered human skeletal muscle microtissue strength. Sci Rep 10, 6918 (2020). https://doi.org/10.1038/s41598-020-62837-8
2. Alexander, M. S., Kawahara, G., Kho, A. T., Howell M. H., Pusack. T. J., Myers, J. A., Montanaro, F., Zon, L. I., Guyon, J. R., Kunkel, L. M. Isolation and transcriptome analysis of adult zebrafish cells enriched for skeletal muscle progenitors. Muscle Nerve. 2011 May; 43(5): 741-750.
https://doi.org/10.1002/mus.21972
3. Araki E, Takayama K, Saito M, Matsuoka H. Separation of viable histamine-producing bacteria from yellowtail meat components by density gradient centrifugation. Biocontrol Sci. 2009 March; 14(1):31-4. doi: 10.4265/bio.14.31. PMID: 19344096.
4. Attree, S. M., Sheffield, E. An evaluation of Ficoll density gradient centrifugation as a method for eliminating microbial contamination and purifying plant protoplasts. Plant Cell Reports 5, 288-291 (1986). https://doi.org/10.1007/BF00269824
5. Belles-Isles M, Roy R, Dansereau G, Goulet M, Roy B, Bouchard J P, Tremblay J P. Rapid selection of donor myoblast clones for muscular dystrophy therapy using cell surface expression of NCAM. Eur J Histochem. 1993; 37(4):375-80. PMID: 7510544.
6. Bischoff, R. and Heintz, C. (1994), Enhancement of skeletal muscle regeneration. Dev. Dyn., 201: 41-54. https://doi.org/10.1002/aja.1002010105
7. Bower N I, Johnston I A. Paralogs of Atlantic salmon myoblast determination factor genes are distinctly regulated in proliferating and differentiating myogenic cells. Am J Physiol Regul Integr Comp Physiol. 2010 June; 298(6):R1615-26. doi: 10.1152/ajpregu.00114.2010. Epub 2010 Apr. 7. PMID: 20375265.
8. Boyum A. Isolation of mononuclear cells and granulocytes from human blood. Isolation of monuclear cells by one centrifugation, and of granulocytes by combining centrifugation and sedimentation at 1 g. Scand J Clin Lab Invest Suppl. 1968; 97:77-89. PMID: 4179068.
9. Cytiva (nee G E Healthcare) (2014), Percoll PLUS/Percoll Instructions, 28-9038-34 A G.
10. Duran, B. O. S., Góes, G. A., Zanella, B. T. T. et al. Ascorbic acid stimulates the in vitro myoblast proliferation and migration of pacu (Piaractus mesopotamicus). Sci Rep 9, 2229 (2019). https://doi.org/10.1038/s41598-019-38536-4
11. J C Engert, E B Berglund, N Rosenthal; Proliferation precedes differentiation in IGF-I-stimulated myogenesis. J Cell Biol 15 Oct. 1996; 135 (2): 431-440. doi: https://doi.org/10.1083/jcb. 135.2.431
12. Fauconneau, B., Paboeuf, G. Effect of fasting and refeeding on in vitro muscle cell proliferation in rainbow trout (Oncorhynchus mykiss). Cell Tissue Res 301, 459-463 (2000). https://doi.org/10.1007/s004419900168
13. Froehlich, J. M., Seiliez, I., Gabillard, J. C., Biga, P. R. Preparation of Primary Myogenic Precursor Cell/Myoblast Cultures from Basal Vertebrate Lineages. J. Vis. Exp. (86), e51354, doi:10.3791/51354 (2014)
14. Greenlee, A. R., Dodson, M. V., Yablonka-Reuveni, Z., Kersten, C. A. and Cloud, J. G. (1995), In vitro differentiation of myoblasts from skeletal muscle of rainbow trout. Journal of Fish Biology, 46: 731-747. https://doi.org/10.1111/j.1095-8649.1995.tb01597.x
15. Han D S, Yang W S, Kao T W. Dexamethasone Treatment at the Myoblast Stage Enhanced C2C12 Myocyte Differentiation. Int J Med Sci 2017; 14(5):434-443. doi:10.7150/ijms.18427.
16. Koumans, J. T. M., Akster, H. A., Dulos, G. J. et al. Myosatellite cells of Cyprinus carpio (Teleostei) in vitro: isolation, recognition and differentiation. Cell Tissue Res 261, 173-181 (1990). https://doi.org/10.1007/BF00329450
17. Langendorf, E. K.; Rommens, P. M.; Drees, P.; Mattyasovszky, S. G.; Ritz, U. Detecting the Effects of the Glucocorticoid Dexamethasone on Primary Human Skeletal Muscle Cells-Differences to the Murine Cell Line. Int. J. Mol. Sci. 2020, 21, 2497
18. Larson A, A, Syverud B, C, Florida S, E, Rodriguez B, L, Pantelic M, N, Larkin L, M: Effects of Dexamethasone Dose and Timing on Tissue-Engineered Skeletal Muscle Units. Cells Tissues Organs 2018; 205:197-207. doi: 10.1159/000490884
19. Latil, M., Rocheteau, P., Châtre, L. et al. Skeletal muscle stem cells adopt a dormant cell state post mortem and retain regenerative capacity. Nat Commun 3, 903 (2012). https://doi.org/10.1038/ncomms1890
20. Lee E J, Pokharel S, Jan A T, et al. Transthyretin: A Transporter Protein Essential for Proliferation of Myoblast in the Myogenic Program. Int J Mol Sci. 2017; 18(1):115. Published 2017 Jan. 8. doi:10.3390/ijms18010115
21. Mansilla E, Martire K, Roque G, Tau J M, Marin G H, et al. (2013) Salvage of Cadaver Stem Cells (CSCs) as a Routine Procedure: History or Future for Regenerative Medicine. J Transplant Technol Res 3: 118. doi:10.4172/2161-0991.1000118
22. Milanesi A, Lee J W, Kim N H, et al. Thyroid Hormone Receptor a Plays an Essential Role in Male Skeletal Muscle Myoblast Proliferation, Differentiation, and Response to Injury. Endocrinology. 2016; 157(1):4-15. doi:10.1210/en.2015-1443
23. Milasincic D J, Calera M R, Farmer S R, Pilch P F. Stimulation of C2C12 myoblast growth by basic fibroblast growth factor and insulin-like growth factor 1 can occur via mitogen-activated protein kinase-dependent and -independent pathways. Mol Cell Biol. 1996; 16(11):5964-5973. doi:10.1128/mcb.16.11.5964
24. Montserrat, N., Sánchez-Gurmaches, J., García de la Serrana, D. et al. IGF-I binding and receptor signal transduction in primary cell culture of muscle cells of gilthead sea bream: changes throughout in vitro development. Cell Tissue Res 330, 503-513 (2007). https://doi.org/10.1007/s00441-007-0507-2
25. Morgan J E. Myogenicity in vitro and in vivo of mouse muscle cells separated on discontinuous Percoll gradients. J Neurol Sci. 1988 June; 85(2):197-207. doi: 10.1016/0022-510×(88)90156-6. PMID: 2838586.
26. Mulvaney, D. R. and Cyrino, J. E. P. (1995), Establishment of channel catfish satellite cell cultures. BAM 5: 65-70.
27. Powell, R. L., Dodson, M. V. and Cloud, J. G. (1989), Cultivation and differentiation of satellite cells from skeletal muscle of the rainbow trout Salmo gairdneri. J. Exp. Zool., 250: 333-338. https://doi.org/10.1002/jez. 1402500314
28. Pretlow T. G., Pretlow T. P. (1977) Separation of Viable Cells by Velocity Sedimentation in an Isokinetic Gradient of Ficoll in Tissue Culture Medium. In: Catsimpoolas N. (eds) Methods of Cell Separation. Biological Separations. Springer, Boston, M A. https://doi.org/10.1007/978-1-4684-0820-1_4
29. Torres-Velarde, J., Bautista-Guerrero, E., Sifuentes-Romero, I., Garcia-Gasca, T., and Garcia-Gasca, G. (2015), A muscle-tissue culture system to study myostatin function in fish. In: Protein biochemistry, synthesis, structure and celular functions. Myostatin. Structure, role in muscle development and health implications.Chapter: A muscle-tissue culture system to study myostatin function in fish.Publisher: Novinka, N Y. E U.Editors: D. Christensen
30. WO2020123876-SYNTHETIC FOOD COMPOSITIONS
31. WO2018227016-EX VIVO MEAT PRODUCTION
32. WO2020149791 ISOLATION AND CULTIVATION OF MUSCLE AND FAT CELLS FROM CRUSTACEANS
33. Yablonka-Reuveni Z, Quinn L S, Nameroff M. Isolation and clonal analysis of satellite cells from chicken pectoralis muscle. Dev Biol. 1987; 119(1):252-259. doi:10.1016/0012-1606(87)90226-0
34. Yablonka-Reuveni Z, Nameroff M. Skeletal muscle cell populations. Separation and partial characterization of fibroblast-like cells from embryonic tissue using density centrifugation. Histochemistry. 1987; 87(1):27-38. doi: 10.1007/BF00518721. PMID: 3038797; PMCID: PMC4096333.
35. Yaffe D. Retention of differentiation potentialities during prolonged cultivation of myogenic cells. Proc Natl Acad Sci USA. 1968; 61(2):477-483. doi:10.1073/pnas.61.2.477
36. Yu M, Wang H, Xu Y, Yu D, Li D, Liu X, Du W. Insulin-like growth factor-1 (IGF-1) promotes myoblast proliferation and skeletal muscle growth of embryonic chickens via the PI3K/Akt signalling pathway. Cell Biol Int. 2015 August; 39(8):910-22. doi: 10.1002/cbin.10466. Epub 2015 Apr. 28. Erratum in: Cell Biol Int. 2016 June; 40(6):727. PMID: 25808997.
37. US20190376026A1-LARGE SCALE CELL CULTURE SYSTEM FOR MAKING MEAT AND ASSOCIATED PRODUCTS
38. Benedetti A, Cera G, De Meo D, Villani C, Bouche1 M, Lozanoska-Ochser B. A novel approach for the isolation and long-term expansion of pure satellite cells based on ice-cold treatment. Skeletal Muscle (2021) 11:7. https://doi.org/10.1186/s13395-021-00261-w
39. Vélez E J, Lutfi E, Jiménez-Amilburu V, Riera-Codina M, Capilla E, Navarro I, Gutiérrez J. IGF-I and amino acids effects through TOR signaling on proliteration and differentiation of gilthead sea bream cultured myocytes. Gen Comp Endocrinol. 2014 Sep. 1; 205:296-304. doi: 10.1016/j.ygcen.2014.05.024. Epub 2014 Jun. 2. PMID: 24882593.
40. Miyata S, Yada T, Ishikawa N, Taheruzzaman K, Hara R, Matsuzaki T, Nishikawa A. Insulin-like growth factor 1 regulation of proliferation and differentiation of Xenopus laevis myogenic cells in vitro. In Vitro Cell Dev Biol Anim. 2017 March:53(3):231-247. doi: 10.1007/s11626-016-0099-9. Epub 2016 Oct. 3. PMID: 27699652.
41. Gabillard J C, Sabin N, Paboeuf G. In vitro characterization of proliferation and differentiation of trout satellite cells. Cell Tissue Res. 2010 December:342(3):471-7. doi: 10.1007/s00441-010-1071-8. Epub 2010 Nov. 18. PMID: 21086139.
42. Duran B O, Fernandez G J, Mareco E A, Moraes L N, Salomão R A, Gutierrez de Paula T, Santos V B, Carvalho R F, Dal-Pai-Silva M. Differential microRNA Expression in Fast- and Slow-Twitch Skeletal Muscle of Piaractus mesopotamicus during Growth. PLOS One. 2015 Nov. 3; 10(11):e0141967. doi: 10.1371/journal.pone.0141967. Erratum in: PLOS One. 2015; 10(12):e0144481. Dal-Pai-Silvca, Maeli [corrected to Dal-Pai-Silva, Maeli]. PMID: 26529415; PMCID: PMC4631509.
43. Millan-Cubillo, A. F., Martin-Perez, M., Ibarz, A. et al. Proteomic characterization of primary cultured myocytes in a fish model at different myogenesis stages. Sci Rep 9, 14126 (2019). https://doi.org/10.1038/s41598-019-50651-w
44. Kong X, Wang X, Li M, Song W, Huang K, Zhang F, Zhang Q, Qi J, He Y. Establishment of myoblast cell line and identification of key genes regulating myoblast differentiation in a marine teleost, Sebastes schlegelii. Gene. 2021 Nov. 15:802:145869. doi: 10.1016/j.gene.2021.145869. Epub 2021 Aug. 2. PMID: 34352298.
45. Alexander M S, Kawahara G, Kho A T, Howell M H, Pusack T J, Myers J A, Montanaro F, Zon L I, Guyon J R, Kunkel L M. Isolation and transcriptome analysis of adult zebrafish cells enriched for skeletal muscle progenitors. Muscle Nerve. 2011 May:43(5):741-50. doi: 10.1002/mus.21972. Epub 2011 Feb. 17. PMID: 21337346; PMCID: PMC3075361.
46. US20080113435-METHOD FOR INDUCING DIFFERENTIATION T O SKELETAL MUSCLE CELLS
47. Hedrick, R. P., McDowell, T. S., Rosemark, R., Aronstein, D. and Lannan, C. N. (1991), Two Cell Lines from White Sturgeon. Transactions of the American Fisheries Society, 120: 528-534. https://doi.org/10.1577/1548-8659(1991)120<0528:TCLFWS>2.3.C O; 2
48. Middlebrooks, B. L., Ellender, R. D., & J. H. Wharton. (1979). Fish Cell Culture: A New Cell Line from Cynoscion nebulosus. In Vitro, 15(2), 109-111. http://www.jstor.org/stable/4292181
49. Fernandez, R. D., Yoshimizu, M., Kimura, T. and Ezura, Y. (1993), Establishment and Characterization of Seven Continuous Cell Lines from Freshwater Fish. Journal of Aquatic Animal Health, 5: 137-147. https://doi.org/10.1577/1548-8667(1993)005<0137:EACOSC>2.3.C O; 2
50. CN104004707A-Pseudosciaena crocea muscle cell line and its establishing method
51. Gignac S J, Vo N T, Mikhaeil M S, Alexander J A, MacLatchy D L, Schulte P M, Lee L E. Derivation of a continuous myogenic cell culture from an embryo of common killifish, Fundulus heteroclitus. Comp Biochem Physiol A Mol Integr Physiol. 2014 September; 175:15-27. doi: 10.1016/j.cbpa.2014.05.002.
52. Lannan, C. N. Fish cell culture: A protocol for quality control. Journal of Tissue Culture Methods 16, 95-98 (1994). https://doi.org/10.1007/BF01404817.
53. Gospodarowicz, D. and Mescher, A. L. (1977), A comparison of the responses of cultured myoblasts and chondrocytes to fibroblast and epidermal growth factors. J. Cell. Physiol., 93: 117-127. https://doi.org/10.1002/jcp.1040930115

1. A method of enhancing the yield of progenitor muscle cells cultured from a sample cell culture, the sample cell culture comprising a plurality of cell types in a culture medium, the method comprising:
(a) plating the sample cell culture on a first surface;
(b) culturing the plated cell culture for sufficient time to allow the progenitor muscle cells to attach onto the first surface;
(c) collecting at least a portion of the culture medium, comprising unattached progenitor muscle cells; and
(d) replating the collected culture medium from (c) onto a further surface to allow the unattached progenitor muscle cells to attach onto the further surface.

2. The method of claim 1, wherein steps (c) and (d) are repeated.

3. The method of claim 1, further comprising washing each plated cell culture with a wash medium, collecting the wash medium, and plating the wash medium onto a second surface.

4. The method of claim 1, comprising prior to (a), obtaining a tissue sample from an organism and filtering the tissue sample to obtain the sample cell culture.

5. The method of claim 4, wherein filtering the tissue sample comprises one or more of:
i) enzymatic dissociation of the sample tissue to extract cells;
ii) density gradient separation to separate debris using a separation medium;
iii) density gradient separation to separate between myogenic cells and fibroblasts;
iv) density gradient separation to separate between myogenic cells and blood cells;
v) density gradient separation having a variable gradient in the range of 1.02 to 1.08 g/mL; and
vi) centrifugation.

6. The method of claim 5, wherein the separation medium is Ficoll-Paque.

7. The method of claim 4, comprising obtaining the tissue sample from the organism post-mortem, and wherein the tissue sample is an animal tissue.

8. (canceled)

9. The method of claim 7, wherein the tissue sample is a seafood animal tissue.

10. The method of claim 9, wherein the tissue sample is a fish tissue.

11. The method of claim 1, wherein the surfaces are coated with laminin with or without poly-lysine, gelatin with or without poly-lysine, collagen I, collagen I V, Matrigel™, or Geltrex™.

12. The method of claim 11, wherein the surfaces are coated with fish gelatin.

13. (canceled)

14. The method of claim 1, wherein the culture medium is supplemented with fibroblast growth factor-2 (FGF-2) based on the fetal bovine serum (FBS) content of the culture medium.

15. The method of claim 14, wherein the culture medium comprises at least 5 ng/ml FGF-2, when the FBS content of the culture medium is less than 10%.

16. The method of claim 1, further comprising culturing and proliferating the attached progenitor muscle cells to a target cell density and/or amount for manufacturing into a food product.

17. A method of producing a food product, the method comprising:
a) extracting cells from a tissue sample obtained from an organism, or from a body part of the organism, post-mortem; and
b) isolating and culturing progenitor muscle cells from the tissue sample into a food product.

18. The method of claim 17, comprising enhancing the yield of progenitor muscle cells from the extracted cells according to the method of claim 1.

19. (canceled)

20. The method of claim 17, comprising obtaining the tissue sample from the organism post-mortem, or from the body part of the organism.

21. The method of claim 17, wherein a) extracting cells comprise one or more of:
i) enzymatic dissociation of the sample tissue to extract cells;
ii) density gradient separation to separate debris using a separation medium;
iii) density gradient separation to separate between myogenic cells and fibroblasts;
iv) density gradient separation to separate between myogenic cells and blood cells;
v) density gradient separation having a variable gradient in the range of 1.02 to 1.08 g/mL; and
vi) centrifugation.

22. The method of claim 17, wherein culturing the progenitor muscle cells comprises culturing the isolated progenitor muscle cells with at least one other type of cells capable of secreting growth stimulatory signals.

23. The method of claim 22, wherein the at least one other type of cells are allogeneic or autologous cells, or xenogeneic cells.

24. (canceled)

25. The method of claim 23, wherein the at least one other type of cells are plant cells.

26. The method of claim 22, wherein the at least one other type of cells comprise non-myogenic cells, such as epithelial cells, nerve cells, fat cells, bone cells, blood cells, immune cells, stem cells, pancreatic, liver and digestive system cells, connective tissue cells, or combinations thereof.

27. The method of claim 22, wherein progenitor muscle cells and the at least one other type of cells are co-cultured and separated by a microporous membrane.

28. The method of claim 22, wherein progenitor muscle cells and the at least one other type of cells are separately cultured and the culture medium from the culturing containing the at least one other type of cells is added to the culture containing the progenitor muscle cells.

29. The method of claim 17, wherein the tissue sample is an animal tissue.

30. The method of claim 29, wherein the tissue sample is a seafood animal tissue.

31. The method of claim 30, wherein the tissue sample is a fish tissue.

32. The method of claim 26, wherein the at least one other type of cells comprise gill epithelial cells, and/or scale cells, and the tissue sample is obtained from a fish.

33. The method of claim 26, wherein the at least one other type of cells comprise liver cells.

34. A method of isolating progenitor muscle cells from a tissue sample comprising separating the progenitor muscle cells from at least one other cell or cellular debris using at least one density gradient.

35. The method of claim 34, comprising separating the progenitor muscle cells using 2 successive density gradients, wherein the density gradient is a Ficoll gradient, a Percoll gradient, or an Optiprep™ gradient.

36. (canceled)

37. (canceled)

38. (canceled)

39. (canceled)

40. The method of claim 34, wherein the progenitor muscle cells are being isolated to be grown into a food product, and wherein the tissue sample is obtained from an organism, or from a body part of the organism, post-mortem, wherein the tissue sample is an animal tissue such as seafood animal tissue.

41. (canceled)

42. (canceled)

43. The method of claim 40, wherein the tissue sample is a fish tissue.

44.-52. (canceled)
